(19) United States
(12) Patent Application Publication
(10) Pub. No.: US 2019/0388124 A1
(43) Pub. Date: Dec. 26, 2019

(54) UNIPLANAR SCREW

(71) Applicant: Astura Medical Inc., Carlsbad, CA (US)

(72) Inventor: Thomas Purcell, Carlsbad, CA (US)

(73) Assignee: Astura Medical Inc., Carlsbad, CA (US)

(21) Appl. No.: 16/450,694

(22) Filed: Jun. 24, 2019

Related U.S. Application Data
(60) Provisional application No. 62/688,467, filed on Jun. 22, 2018.

(51) Int. Cl. A61B 17/70; A61B 17/86

(52) U.S. Cl. CPC A61B 17/7038; A61B 17/8625; A61B 17/8605; A61B 17/7032

(57) ABSTRACT

A uniplanar screw assembly having a pedicle screw with a threaded shaft and spherical head with a circumferential groove; a U-shaped tulip having a spherical bottom sized to receive the spherical head of the pedicle screw, a bottom opening for the threaded shaft to go through, and diametrically opposed pin openings; two pins sized for insertion in the pin holes of the U-shaped tulip being configured to slideingly engage the circumferential groove in the spherical head; wherein the engagement of the diametrically opposed pins and circumferential groove limits a lateral motion of the pedicle screw with respect to the U-shaped tulip in a plane that pins are symmetric to, while allowing a rotational motion between the pins and groove so that the pedicle screw may rotate with respect to the U-shaped tulip.

### CROSS-REFERENCE TO RELATED APPLICATIONS

This application claims the benefit of U.S. Provisional Application No. 62/688,467 filed Jun. 22, 2018, which is incorporated herein by reference.

### FIELD

The present invention relates generally to the field of surgery, and more specifically, to a uniplanar screw for use with a spinal fixation system.

### BACKGROUND

Certain spinal conditions, including a fracture of a vertebra and a herniated disc, indicate treatment by spinal immobilization. Several methods of spinal joint immobilization are known, including surgical fusion and the attachment of pins and bone plates to the affected vertebras. One known device is a stabilization rod interconnecting the two or more pedicle screws to stabilize the vertebras spanned by the screws. The screw includes a channel for receiving the stabilization rod and a nut to apply compressive force between the rod and the screw head to firmly fix the rod between the spanned vertebras and thus stabilize the spinal vertebrae.

The angle at which the anchor screws extend from the vertebra pedicle is dictated by the spinal curvature, the orientation of individual vertebra within the spine, and the surgeon's placement of the screw within the pedicle. The rod-receiving channels are typically not collinear nor coplanar and the rod must be shaped or contoured during the implantation procedure to fit within the channels along the spinal column. The prior art systems allow the coupling unit to pivot with respect to the screw.

It may be desirable to use an anchor that provides a range of motion in only one plane.

### SUMMARY

The present invention allows a first range of motion in all directions, but also provides an extended range of motion in the medial-lateral-inferior direction (head-to-toe). This extended range of motion, as compared to the prior art, allows the surgeon additional freedom in locating the screws and eases the assembly process by reducing the requirement for rod contouring.

### BRIEF DESCRIPTION OF THE DRAWINGS

FIG. 1 is a view showing a one embodiment of a uniplanar screw assembly.

FIG. 2 shows the single plane motion of the screw of the uniplanar screw assembly.

FIG. 3 is a cross-sectional view of the U-shaped tulip in the uniplanar screw assembly.

FIG. 4 is a cross-sectional view showing the unique design of the Tulip/Screw interface.

FIG. 5A shows a Tulip/Screw interface that is designed to restrict the pedicle screw to planar motion or unilateral motion.

FIG. 5B shows a Tulip/Screw interface that is designed to allow rotational motion between the pedicle screw and U-shaped tulip.

FIG. 6 is a perspective view of the pedicle screw.

FIG. 7 is a cross-sectional view of the upper portion of the pedicle screw.

FIG. 8 is a perspective view of the U-shaped tulip.

FIG. 9 is a cross-section view of the U-shaped tulip.

FIG. 10 is a perspective view of the bushing.

FIG. 11 is a cross-section view of the bushing.

FIG. 12A shows a standard U-shaped tulip.

FIG. 12A shows a high top U-shaped tulip.

### DETAILED DESCRIPTION

The uniplanar screw of the present invention may be used with a pedicle screw fixation system (PSFS) for a variety of conditions that affect the thoracic and lumbar spine. In cases in which the posterior elements are fractured, a pedicle screw fixation system using uniplanar screws offers an excellent means of stabilizing a specific spinal segment. Due to the biomechanical advantages of a pedicle screw fixation system, a spine stabilized with the uniplanar screws may not require the use of postoperative bracing and thus enhance rehabilitation. The pedicle screw fixation system can be used in patients with deformities that require aggressive coronal and sagittal plane correction, as well as deformities that require osteotomies. The pedicle screw fixation system can be used in patients suffering from degenerative lumbar disease or pathological processes, and in correction of spinal deformity.

The pedicle screw fixation system is intended to provide immobilization and stabilization of spinal segments in skeletally mature patients as an adjunct to fusion in the treatment of the following acute and chronic instabilities or deformities of thoracic, lumbar, and sacral spine: degenerative spondylolisthesis with objective evidence of neurological impairment, fracture, dislocation, deformities or curvatures (i.e. scoliosis, kyphosis, and/or lordosis), spinal tumor, pseudarthrosis and failed previous fusion.

The pedicle screw fixation system may also be used for non-cervical pedicle screw fixation for the following indications: severe spondylolisthesis (grades 3 and 4) of the L5-S1 vertebra in skeletally mature patients receiving fusion by autogenous bone graft having implants attached to the lumbar and sacral spine (L3 to sacrum) with removal of the implants after the attainment of a solid fusion. It is also intended for the following indications: trauma (i.e. fracture or dislocation); spinal stenosis; deformities or curvatures (i.e. scoliosis, kyphosis, and/or lordosis); tumor; pseudoarthrosis; failed previous fusion; and adolescent scoliosis.

The pedicle screw fixation system with uniplanar screws is a top loading thoracolumbar, sacral, and iliac fixation system implanted from the posterior approach and designed to provide fixation during the fusion process. The system is composed of preassembled uniplanar screws, rods, crosslinks, and rod connectors. The system is supported by a comprehensive set of instruments to install the implants within the system.

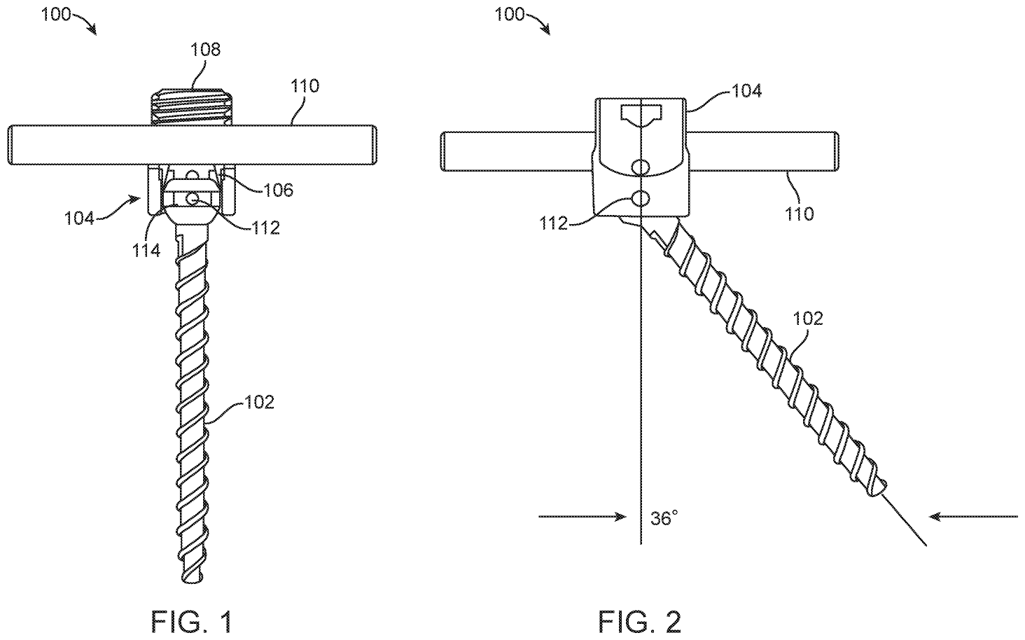

Referring now to FIGS. 1 and 2, a uniplanar screw assembly 100 is shown that includes a pedicle screw 102, a U-shaped tulip 104, a bushing 106, and a locking device 108 configured to hold a rod 110. The interface between the U-shaped tulip 104 and pedicle screw 102 (“Tulip/Screw interface”) uses a unique design that allows the pedicle screw 102 rotate with respect to the U-shaped tulip 104, while at the same time limiting the motion between the U-shaped tulip 104 and pedicle screw 102 to a single plane. The benefit of this design is that it allows the pedicle screw 102 to be inserted to the desired depth in the bone, and then the U-shaped tulip 104 may be adjusted to align with the rod 110. Then the locking device 108, such as a set screw, may be tightened and torqued into the rod 110, which contacts the bushing 106, which contacts the screw head 116 to create a frictional lock.

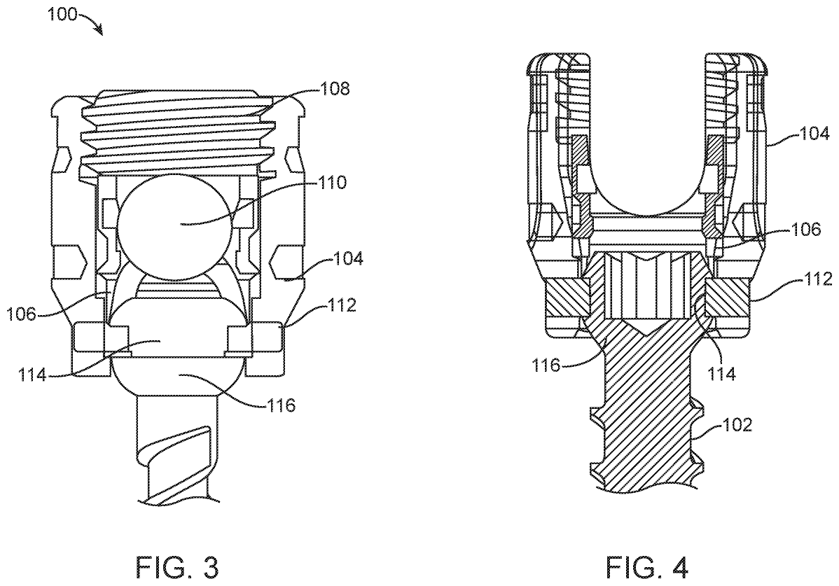

FIG. 3 is a cross-sectional view of the U-shaped tulip 104 of the uniplanar screw assembly 100, and FIG. 4 is a cross-sectional view showing the unique design of the Tulip/Screw interface. In the embodiment shown, diametrically opposed pins 112 in the U-shaped tulip 104 engage a circumferential groove 114 in the screw head 116 to limit the motion of the pedicle screw 102 in the plane that pins 112 are symmetric to (i.e. uniplanar motion), while still allowing a rotational motion between the pins 112 and groove 114 so that the pedicle screw 102 may rotate with respect to the U-shaped tulip 104 during pedicle screw 102 insertion into the bone.

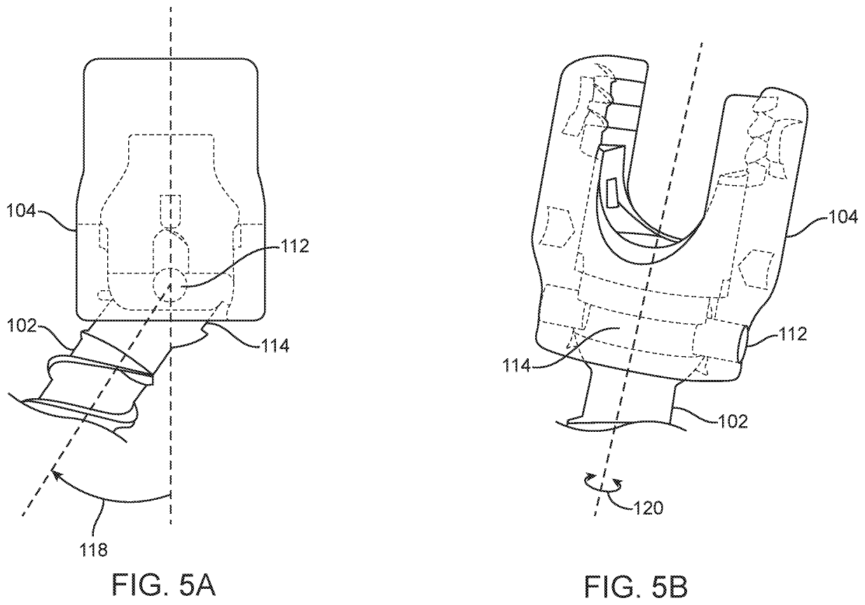

FIG. 5A shows a Tulip/Screw interface that is designed to restrict the pedicle screw to planar motion or unilateral motion 118 in a medial-lateral direction. While the embodiment shown limits motion to the medial-lateral direction, other embodiments of the screw may limit motion in other directions, depending on where the screw is used. In the embodiment shown, the Tulip/Screw interface allows a maximum angular variability of ±36° (shown in FIG. 2), thereby reducing the necessity of rod bending. In other embodiments, the Tulip/Screw interface may be configured for more or less angular variability, depending on the uniplanar screw application.

FIG. 5B shows a Tulip/Screw interface that is designed so that the pins 112 can slide in the groove 114 and allow rotational motion 120 between the pedicle screw 102 and U-shaped tulip 104 during insertion into the bone. The benefit of this is that it allows the pedicle screw to be inserted to the desired depth and then the tulip can be adjusted to align with the rod.

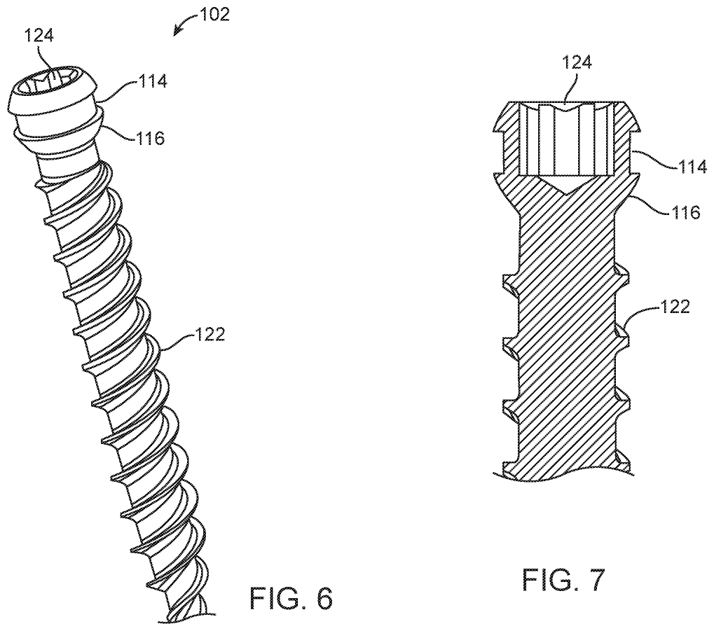

FIG. 6 is a perspective view of the pedicle screw 102 and FIG. 7 is a cross-sectional view of the upper portion of the pedicle screw 102. The pedicle screw 102 includes the spherical head 116 with circumferential groove 114 and a threaded shaft 122 configured for insertion into bone. The spherical head 116 may further include a recess or slot 118 on the top for use with a tool used to drive the pedicle screw into the bone or other tools. The threaded shaft 122 should be capable of being placed quickly with minimal effort and that resists pullout and may include different lead designs.

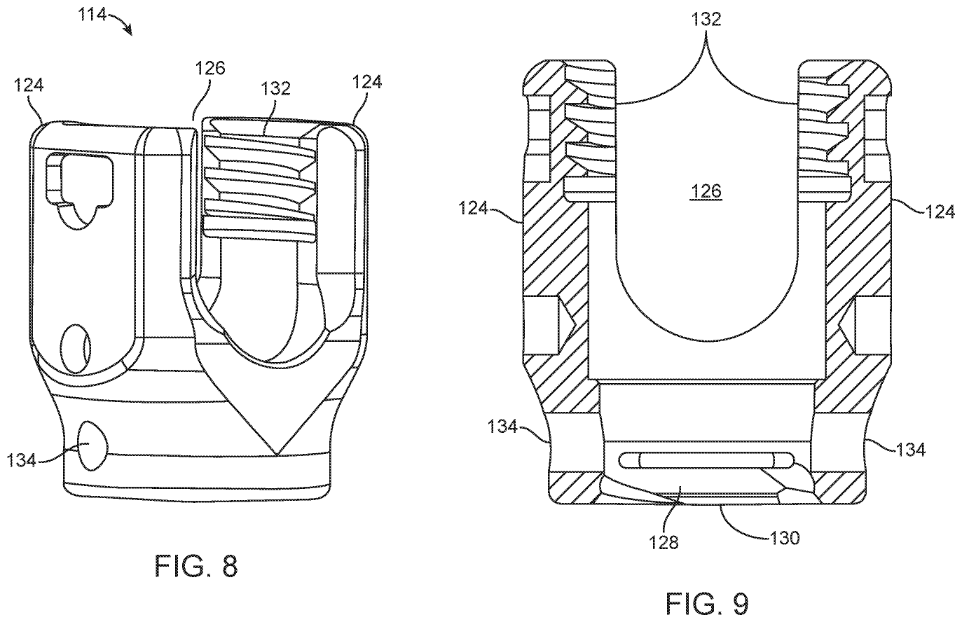

FIG. 8 is a perspective view of the U-shaped tulip 104 and FIG. 9 is a cross-section view. The U-shaped tulip 104 includes a side wall 124 having U-shaped openings or slots 126 sized to receive a rod 110, a spherical bottom 128 sized to receive the spherical head 116 of the pedicle screw 102, and a bottom opening or hole 130 for the threaded shaft 122 to go through. The interior of the U-shaped opening 126 of the U-shaped tulip may be threaded 132 to accept the locking Device 108 to securely clamp the rod 110 to the top of the pedicle screw 102. The U-shaped tulip 104 further includes diametrically opposed pin openings 134 sized to receive pins 112. As discussed above, the pins 112 configured to engage the circumferential groove 114 and restrict screw motion to the medial-lateral direction.

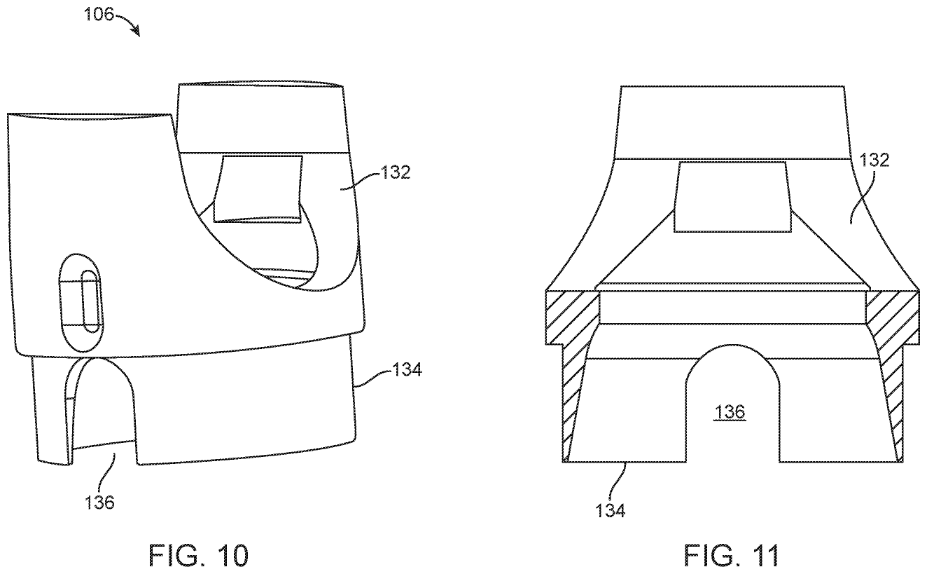

FIG. 10 is a perspective view of the bushing 106 and FIG. 9 is a cross-section view. The bushing 106 is configured to fit within the U-shaped tulip 104 adjacent side walls 124 to distribute the longitudinal forces exerted on the pedicle screw 102. The bushing 106 includes an upper portion having a rod engagement surface, such as a concave surface 132 defining a seat or saddle for the rod 110. The bushing 106 includes lower portion having a slotted skirt 134 with pin slots 136. The slotted skirt 134 can be configured to provide a press fitment about the screw head 116 so that the pedicle screw 102, U-shaped tulip 104 and bushing 106 can be locked in place.

As discussed above, the U-shaped tulip 104 includes a bottom opening or hole 130 that is dimensioned to allow the threaded shaft 122 to pass therethrough, but the dimension of the spherical bottom 128 is smaller than the spherical head 116, and thereby prevents passage of the spherical head 116 therethrough, as illustrated in figures. Pins 112 are inserted into the pin holes 134 so that they engage the circumferential groove 114 of the pedicle screw 102. The circumferential groove 114 is configured and dimensioned to receive the protruding ends of the pins 112, to allow screw 102 rotation in the U-shaped tulip 104 and restrict screw 102 lateral motion to the medial-lateral direction. In the embodiment shown, the Tulip/Screw interface allows a maximum angular variability of ±36°, thereby reducing the necessity of rod bending. In other embodiments, the Tulip/Screw interface may be configured for more or less angular variability, depending on the uniplanar screw application.

To lock the components in place, the locking device 108 is advanced within the U-shaped tulip 104 to contact the rod 110, which forces the rod 110 against the concave surface 132 of the bushing 106, which in turn may force the slotted skirt 134 against the inner walls U-shaped tulip 104 and spherical head 116 of the pedicle screw 102. Prior to final tightening of the locking device 108, the slotted skirt 134 may frictionally engage the spherical head 116 of the pedicle screw 102 to resist movement of the U-shaped tulip 104 with respect to the pedicle screw 102. After tightening of the set screw, the frictional force between the bushing 106 and the spherical head 116 of the pedicle screw 102 may be sufficient to lock the pedicle screw 102 in place with respect to the U-shaped tulip 104. In some examples, bushing 106 may be omitted, and the locking device 108 may force the rod 110 directly against the spherical head 116 of the pedicle screw 102 to secure the pedicle screw 102 in place.

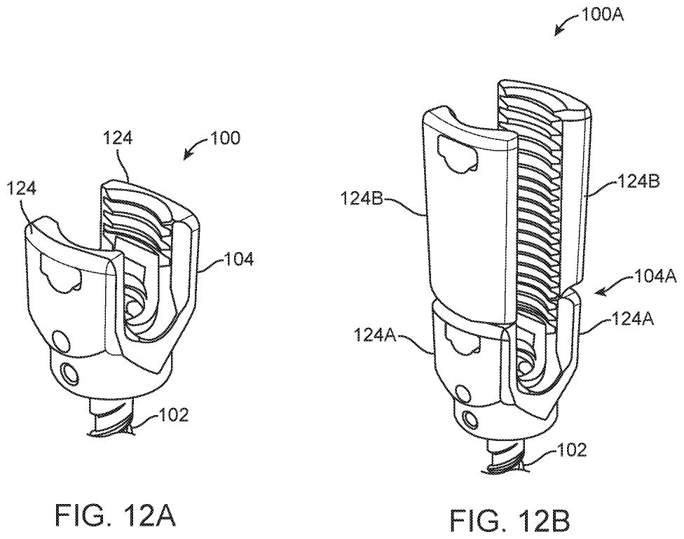

The uniplanar screw acts as an anchor point for other hardware when installed into the spinal anatomy. The uniplanar screw may include thread options that allow for fixation based on surgeon preference and anatomy. The U-shaped tulip may also include options that allow for a uniplanar screw 100 for standard medial-lateral motion (Standard), shown in FIG. 12A, and a uniplanar screw 100A having a U-shaped tulip 104A with extended sidewalls 124A for spondylolisthesis reduction (High Top), shown in FIG. 12B. The extended sidewalls 124A include break-away top portions 124B that are removed or broken off in that once the assembly is locked with the locking device 108. The pedicle screw may also be cannulated for installation using a guide wire. The uniplanar screw may have varying diameters and lengths of Standard, High Top, and Cannulated configurations. The uniplanar screw may be made of titanium alloy.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A uniplanar screw assembly comprising:
a screw having a shaft and spherical head with a circumferential groove;
a tulip having a spherical bottom sized to receive the spherical head of the screw and diametrically opposed pin openings; and
pins sized for insertion in the pin holes of the tulip being configured to slideingly engage the circumferential groove in the spherical head;
wherein the engagement of the diametrically opposed pins and circumferential groove limits a lateral motion of the screw with respect to the tulip to a plane while allowing a rotational motion of the screw with respect to the tulip.

2. The uniplanar screw assembly of claim 1, wherein the limited lateral motion is in a plane that pins are symmetric to.

3. The uniplanar screw assembly of claim 1, wherein the screw is a pedicle screw with a threaded pedicle shaft.

4. The uniplanar screw assembly of claim 3, wherein the tulip includes a bottom opening for the threaded shaft to go through.

5. The uniplanar screw assembly of claim 1, further comprising a locking device configured to lock the spherical head of the screw against the spherical bottom of the tulip in a desired position.

6. The uniplanar screw assembly of claim 1, wherein the tulip is a U-shaped tulip having side walls with U-shaped openings sized to receive a rod.

7. The uniplanar screw assembly of claim 6, further comprising a bushing configured to fit within the U-shaped tulip, the bushing having an upper portion with a concave surface defining a seat or saddle sized for the rod and a lower portion having a skirt configured to engage the spherical head of the screw, the bushing being located between and in contact with the rod and the spherical head.

8. The uniplanar screw assembly of claim 7, further comprising a locking device coupled to the U-shaped tulip being configured to apply pressure to a rod within the U-shaped openings which in turn pushes downwardly on the bushing to apply pressure to lock the spherical head of the screw against the spherical bottom of the U-shaped tulip.

9. The uniplanar screw assembly of claim 8, wherein the interior of the U-shaped opening of the U-shaped tulip may be threaded to accept a set screw configured to be tightened and torqued into the rod, which contacts the bushing, which contacts the screw head to create a frictional lock.

10. A uniplanar screw assembly comprising:
a pedicle screw having a threaded shaft and spherical head with a circumferential groove;
a tulip having side walls with openings sized to receive a rod, a spherical bottom sized to receive the spherical head of the pedicle screw, a bottom opening for the threaded shaft to go through, and diametrically opposed pin openings;
two pins sized for insertion in the pin holes of the tulip being configured to slideingly engage the circumferential groove in the spherical head;
wherein the engagement of the diametrically opposed pins and circumferential groove limits a lateral motion of the pedicle screw with respect to the tulip in a plane that pins are symmetric to, while allowing a rotational motion between the pins and groove so that the pedicle screw may rotate with respect to the tulip.

11. The uniplanar screw assembly of claim 10, further comprising a locking device coupled to the tulip being configured to apply pressure to a rod within the openings which in turn pushes downwardly to apply pressure to lock the spherical head of the pedicle screw against the spherical bottom of the tulip.

12. The uniplanar screw assembly of claim 11, further comprising a bushing configured to fit within the tulip, the bushing an upper portion having a rod engagement surface and a lower portion having a skirt, the bushing being located between and in contact with the rod and the spherical head of the pedicle screw.

13. The uniplanar screw assembly of claim 12, wherein the interior of the U-shaped opening of the U-shaped tulip may be threaded to accept a set screw configured to be tightened and torqued into the rod, which contacts the bushing, which contacts the screw head to create a frictional lock.

14. A uniplanar screw assembly comprising:
a pedicle screw having a threaded shaft and spherical head with a circumferential groove;
a U-shaped tulip having side walls with U-shaped openings sized to receive a rod, a spherical bottom sized to receive the spherical head of the pedicle screw, a bottom opening for the threaded shaft to go through, and diametrically opposed pin openings;
two pins sized for insertion in the pin holes of the U-shaped tulip being configured to slideingly engage the circumferential groove in the spherical head;
wherein the engagement of the diametrically opposed pins and circumferential groove limits a lateral motion of the pedicle screw with respect to the U-shaped tulip in a plane that pins are symmetric to, while allowing a rotational motion between the pins and groove so that the pedicle screw may rotate with respect to the U-shaped tulip; and
a locking device coupled to the U-shaped tulip being configured to apply pressure to a rod within the U-shaped openings which in turn pushes downwardly to apply pressure to lock the spherical head of the pedicle screw against the spherical bottom of the U-shaped tulip so as to fix the angular position of the pedicle screw relative to the U-shaped tulip when in an assembled configuration.

15. The uniplanar screw assembly of claim 14, further comprising a bushing configured to fit within the U-shaped tulip, the bushing an upper portion having a concave surface defining a seat or saddle for the rod and a lower portion having a skirt, the bushing being located between and in contact with the rod and the spherical head of the pedicle screw.
